(19) United States
(12) Patent Application Publication
(10) Pub. No.: US 2012/0267819 A1
(43) Pub. Date: Oct. 25, 2012

(54) ANTIOXIDANT DOPING OF CROSSLINKED POLYMERS AT HIGH PRESSURES

(75) Inventor: Jordan H. FREEDMAN, Fort Wayne, IN (US)

(73) Assignee: Biomet Manufacturing Corp., Warsaw, IN (US)

(21) Appl. No.: 13/092,705

(22) Filed: Apr. 22, 2011

(51) Int. Cl. B28B 11/08; A61F 2/30; C08K 5/1545

(52) U.S. Cl. 264/162; 524/110; 523/113

(57) ABSTRACT

Methods for an antioxidant doped polymer in the form of an implant bearing component. The process includes: (a) contacting a crosslinked polymer with a liquid composition comprising an antioxidant, to provide an intermediate polymer with the antioxidant on its surface; and (b) homogenizing the intermediate polymer by raising the pressure to increase the onset melting temperature of the polymer, and then heating above the ambient onset temperature but below the raised onset melting point of the polymer.

### INTRODUCTION

The present technology relates to antioxidant doping of crosslinked polymers. Specifically, the technology relates to processes for incorporating antioxidant materials into crosslinked polymers for use in medical implants.

Crosslinked polymers such as ultra high molecular weight polyethylene (UHMWPE) have found wide application in medical implants as bearing components. The crosslinked polymers exhibit favorable wear properties and have good bio-compatibility. In addition to good wear properties, it is also important to provide materials that resist oxidation so that the life of the material in the body can be increased.

A variety of techniques has been used to increase the oxidation stability of crosslinked materials such as UHMWPE. In some, a series of heat treatment or annealing steps are performed on the crosslinked material to decrease or eliminate the free radicals induced by the crosslinking. It is generally known that to incorporate an antioxidant material into a polymer, it is necessary to perform the annealing step at a temperature below the crystalline melting point in order to not destroy the strength of the polymeric material. Annealing at the lower temperature increases the time it takes to diffuse the polymer with an antioxidant, such as Vitamin E, directly into the polymer.

Improved methods of doping a crosslinked polymer with an antioxidant followed by annealing of the antioxidants into crosslinked polymers in order to provide a doped crosslinked polymer would be a significant advance.

### SUMMARY

Methods have been developed to diffuse an antioxidant material such as Vitamin E directly into a crosslinked polymer material at an elevated pressure. The elevated pressure in turn raises the normal melting temperature, which allows the crosslinked polymer material to be heated to a higher temperature while still avoiding melting or the onset of melting. The higher temperature decreases the diffusion time.

An advantageous feature of the methods is that antioxidant doped polymer articles are produced with a faster cycle time, since the homogenizing step can be carried out for a shorter time at the elevated temperature. In particular, in various embodiments, a crosslinked UHMWPE is doped with Vitamin E. After doping, the doped UHMWPE, which contains Vitamin E on its surface, is subjected (preferably in an inert atmosphere) to an elevated pressure sufficient to raise the melting point of the UHMWPE. The doped UHMWPE is then homogenized at the elevated pressure by heating at a temperature that is above the normal onset melting temperature of the UHMWPE. (Such “onset” temperatures are described further herein. Throughout, “normal” melting temperature and “normal” onset melting temperature mean the respective values of the UHMWPE material at ambient or atmospheric pressure.) Even though the UHMWPE is heated during the homogenizing step at a temperature above its normal onset melting temperature, it does not melt or degrade because the high pressure used during the homogenizing step raises the melting temperature of the crosslinked UHMWPE. By carrying out homogenizing at a temperature higher than the normal onset melting temperature, the time for diffusion can be lowered relative to prior art methods where homogenizing is carried out below the lower “normal” temperatures. In various embodiments, the elevated pressure is from 10 MPa to 500 MPa. The disclosed methods provide materials that are, for example, useful and suitable as bearing components for implantation into the human body.

### DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. A non-limiting discussion of terms and phrases intended to aid understanding of the present technology is provided at the end of this Detailed Description.

The present technology provides a method of preparing an antioxidant doped crosslinked polymer. In various embodiments, the methods are characterized by a series of doping, homogenizing, and cooling steps. Methods include a final machining step after the crosslinked polymer, such as an UHMWPE, is doped with antioxidant and homogenized at an elevated pressure in an inert atmosphere. In this way, bearing components and other UHMWPE articles contain antioxidants such as Vitamin E throughout the polymer of the component or material and are prepared by a method that decreases the time it takes to diffuse the antioxidant through the UHMWPE.

In various embodiments, the present technology provides polymeric materials such as UHMWPE suitable for use as bearing components in medical implants. Such implants may be used in hip replacements, knee replacements, and the like, as further discussed herein. The polymeric material is crosslinked to increase its wear properties. The crosslinked polymeric material is treated in a doping step by contacting with and in-diffusion of antioxidant compositions that serve to eliminate or trap free radicals in the material. The material is then exposed to an elevated pressure, preferably in an inert atmosphere, and heated at an elevated temperature which lowers the time for diffusion of the antioxidant compositions through the material. As a result, the oxidation properties of the crosslinked material are improved. Antioxidants include, without limitation, Vitamin E, α-tocopherols, retinoids, and the like.

In one embodiment, a method of preparing an antioxidant doped polymer includes the steps of: (a) providing a crosslinked polymer, (b) contacting the crosslinked polymer with a liquid composition comprising an antioxidant, to provide an intermediate polymer with antioxidant on its surface, and then (c) homogenizing the intermediate polymer by (1) exposing the intermediate polymer to an elevated pressure in an inert atmosphere, wherein the elevated pressure is high enough to raise the onset melting temperature of the intermediate polymer to an elevated onset melting temperature above its ambient onset melting temperature determined at atmospheric pressure, and (2) heating the intermediate polymer at the elevated pressure to a temperature above the ambient onset melting temperature but below the elevated onset melting temperature for a time sufficient to achieve diffusion of the antioxidant from the surface into the interior of the intermediate polymer and produce a doped polymer, and then (d) cooling the doped polymer after the homogenizing. The doped polymer can then be further processed to make a bearing component for a medical implant.

As further discussed herein, heating and pressure steps are discussed in reference to an “onset melting temperature.” It is well known that polymers do not melt at a single temperature or at a sharp temperature range for the reason that they are not pure substances. Instead, most polymers are made of a number of different chemical species, all of which if pure could be characterized by a single melt temperature, but when combined tend to melt over a wide range of temperatures. The resulting polydispersity also leads to entropy effects. The result is that polymers “melt” over a broad temperature range.

In a well known phenomenon, as the temperature of a polymer is raised in a calorimetric experiment (such as the well known DSC, or differential scanning calorimetry), there is an onset of an endotherm in the DSC trace at what will be called here an onset melting temperature. Several degrees above the onset melting temperature, the polymer usually exhibits a peak in the DSC curve. The top of the peak can also be considered a melting temperature, but it can be several degrees higher than the onset melting temperature. For example, some crystalline UHMWPE exhibits a peak melting temperature (at atmospheric pressure) of 140-144° C., but an onset melting temperature of about 132-133° C. For best results, homogenizing in the current technology is carried out below the onset melting temperature.

The melting transition of a polymer increases in response to a high applied pressure. This affects both the onset melting temperature and the peak melting temperature as measured in the DSC experiment, which are shifted to higher values at the high pressures. Essentially, both the onset and the peak temperatures are raised together, to approximately the same extent. Advantageously, the onset melting temperature of a polymer such as UHMWPE can be increased by 5° C. or more by applying pressures easily reachable by modern pressurization equipment. This characteristic is exploited in the current technology to raise the temperatures at which a pressurized polymer can be heated without exceeding the onset melting temperature. Specifically, the phenomenon is used to achieve homogenizing temperatures for antioxidant doped polymers that are not available by heating at ambient pressures.

In various aspects of the current technology, heating a polymer to a temperature above a “melting temperature” or above a “melting point” is to be avoided for the reason that doing so would at least partially melt the polymer and lead to a dimunition or degradation of some desired property. Accordingly, in some embodiments, heating may be to a temperature below a melting point or melting temperature (the terms are interchangeable, except where the context might require otherwise). For precision, processes of the present technology generally reflect a desirability to heat below the “onset” melting temperature.

Specific exemplary methods for UHMWPE include: (a) doping a crosslinked UHMWPE by contacting it with a liquid composition comprising Vitamin E to produce a UHMWPE intermediate with Vitamin E on its surface; (b) homogenizing the intermediate UHMWPE by (1) subjecting the intermediate UHMWPE with Vitamin E on its surface to a pressure above 10 MPa, at which pressure the intermediate UHMWPE has an elevated onset melting temperature (i.e., an onset melting temperature higher than the UHMWPE's onset melting temperature at atmospheric pressure), and (2) heating the intermediate UHMWPE at the elevated pressure to a temperature greater than its normal onset melting temperature at atmospheric pressure but less than the elevated onset melting temperature for a time sufficient to achieve diffusion of Vitamin E from the surface into the interior of the intermediate UHMWPE; and then (c) cooling the UHMWPE after homogenizing.

In another embodiment, a process for making an artificial joint component includes the further step of: (d) machining the joint component from the doped UHMWPE or other crosslinked polymer treated by steps the doping, homogenizing, and cooling steps (a) through (c) recited above.

In a particular embodiment, the present technology provides a method of making an oxidation resistant UHMWPE by the methods described herein where the doping and homogenizing are repeated at least once. In an exemplary embodiment, a method involves exposing a polymeric material to an antioxidant composition comprising Vitamin E. The method involves exposing a polymer (such as crosslinked UHMWPE) to a composition comprising Vitamin E at a temperature below the crystalline melting point and preferably below the onset melting temperature of the UHMWPE. Thereafter, the UHMWPE is removed from exposure to the Vitamin E composition and is homogenized by heating it to a temperature at least 5° C. above its normal onset melting temperature while exposing the polymer to an elevated pressure that raises the (onset) melting temperature above the temperature to which it is being heated. The exposing and homogenizing steps can be repeated at least once to enhance diffusion of the antioxidant into the interior of the bulk polymer.

The individual steps of doping, homogenizing and cooling outlined above are carried out in the order recited in the various embodiments, although it will be appreciated that additional steps may be performed in other sequences in various embodiments, and that specific manufacturing processes may employ methods where individual steps are performed in whole or in part at the same time. Various parameters of each of the steps are described below. It is intended that any of the parameters described for individual steps can be combined in processes to make suitable bearing implant components.

### Polymers

Preferred polymers for use in the methods of this technology include those that are wear resistant, have chemical resistance, resist oxidation, and are compatible with physiological structures. In various embodiments, the polymers are polyesters, polymethylmethacrylate, nylons or polyamides, polycarbonates, and polyhydrocarbons such as polyethylene and polypropylene. High molecular weight and ultra high molecular weight polymers are preferred in various embodiments. Non-limiting examples include high molecular weight polyethylene, ultra high molecular weight polyethylene (UHMWPE), and ultra high molecular weight polypropylene. In various embodiments, the polymers have molecular ranges from approximate molecular weight range from about 400,000 to about 10,000,000.

UHMWPE is used in joint replacements because it possesses a low co-efficient of friction, high wear resistance, and compatibility with body tissue. UHMWPE is available commercially as bar stock or blocks that have been compression molded or ram extruded. Commercial examples include the GUR® series from Ticona. A number of grades are commercially available having molecular weights in the preferred range described above. UHMWPE useful herein includes materials in flake form as are commercially available from a number of suppliers. In various embodiments, UHMWPE starting materials are produced from the powdered UHMWPE polymer by methods known in the art.

In one embodiment, the UHMWPE is provided in the form of cylinders or rods having a diameter of 1 to 4 inches. Preferred processes for producing a UHMWPE starting material are described in U.S. Pat. No. 5,466,530, England et al., issued Nov. 14, 1995 and U.S. Pat. No. 5,830,396, Higgins et al., issued Nov. 3, 1998, the disclosures of which are incorporated by reference.

### Crosslinking

In various embodiments, the polymer material provided can be crosslinked by a variety of chemical and radiation methods. Chemical crosslinking may be accomplished by combining a polymeric material with a crosslinking chemical and subjecting the mixture to temperature sufficient to cause crosslinking to occur. For example, the chemical crosslinking can be accomplished by molding a polymeric material containing the crosslinking chemical. The molding temperature is the temperature at which the polymer is molded, which may be at or above the melting temperature of the polymer.

If the crosslinking chemical has a long half-life at the molding temperature, it will decompose slowly, and the resulting free radicals can diffuse in the polymer to form a homogeneous crosslinked network at the molding temperature. Thus, the molding temperature is also preferably high enough to allow the flow of the polymer to occur to distribute or diffuse the crosslinking chemical and the resulting free radicals to form the homogeneous network. For UHMWPE, a preferred molding temperature is between about 130° C. and 220° C. with a molding time of about 1 to 3 hours. In a non-limiting embodiment, the molding temperature and time are 170° C. and 2 hours, respectively.

The crosslinking chemical may be any chemical that decomposes at the molding temperature to form highly reactive intermediates, such as free radicals, that react with the polymers to form a crosslinked network. Examples of free radical generating chemicals include peroxides, peresters, azo compounds, disulfides, dimethacrylates, tetrazenes, and divinylbenzene. Examples of azo compounds include: azobis-isobutyronitrile, azobis-isobutyronitrile, and dimethylazodi-isobutyrate. Examples of peresters include t-butyl peracetate and t-butyl perbenzoate.

In various embodiments, the polymer is crosslinked by treating it with an organic peroxide. Suitable peroxides include 2,5-dimethyl-2,5-bis(tert-butylperoxy)-3-hexyne (Lupersol 130, Atochem Inc., Philadelphia, Pa.); 2,5-dimethyl-2,5-di-(t-butylperoxy)-hexane; t-butyl α-cumyl peroxide; di-butyl peroxide; t-butyl hydroperoxide; benzoyl peroxide; dichlorobenzoyl peroxide; dicumyl peroxide; di-tertiary butyl peroxide; 2,5-dimethyl-2,5-di(peroxy benzoate)hexyne-3; 1,3-bis(t-butyl peroxy isopropyl) benzene; lauroyl peroxide; di-t-amyl peroxide; 1,1-di-(t-butylperoxy) cyclohexane; 2,2-di-(t-butylperoxy)butane; and 2,2-di-(t-amylperoxy) propane. A preferred peroxide is 2,5-dimethyl-2,5-bis(tert-butylperoxy)-3-hexyne. The preferred peroxides have a half-life of between 2 minutes to 1 hour; and more preferably, the half-life is between 5 minutes to 50 minutes at the molding temperature. Generally, between 0.2 to 5.0 wt % of peroxide is used; more preferably, the range is between 0.5 to 3.0 wt % of peroxide; and most preferably, the range is between 0.6 to 2 wt %.

The peroxide can be dissolved in an inert solvent before being added to the polymer powder. The inert solvent preferably evaporates before the polymer is molded. Examples of such inert solvents are alcohol and acetone.

For convenience, the reaction between the polymer and the crosslinking chemical, such as peroxide, can generally be carried out at molding pressures. Generally, the reactants are incubated at molding temperature, between 1 to 3 hours, and more preferably, for about 2 hours.

The reaction mixture is preferably slowly heated to achieve the molding temperature. After the incubation period, the crosslinked polymer is preferably slowly cooled down to room temperature. For example, the polymer may be left at room temperature and allowed to cool on its own. Slow cooling allows the formation of a stable crystalline structure.

The reaction parameters for crosslinking polymers with peroxide, and the choices of peroxides, can be determined by one skilled in the art. For example, a wide variety of peroxides are available for reaction with polyolefins, and investigations of their relative efficiencies have been reported. Differences in decomposition rates can be an important factor in selecting a particular peroxide for an intended application. For example, UHMWPE has also been reported. UHMWPE can be crosslinked in the melt at 180° C. by means of 2,5-dimethyl-2,5-di-(tert-butylperoxy)-hexyne-3.

In various embodiments, crosslinking is accomplished by exposing a polymeric material to irradiation. Non-limiting examples of irradiation for crosslinking the polymers include electron beam, x-ray, and γ-irradiation. In various embodiments, γ-irradiation is preferred because the radiation readily penetrates the polymer material. Electron beams can also be used to irradiate the polymer material. With e-beam radiation, the penetration depth depends on the energy of the electron beam, as is well known in the art.

For gamma (γ) irradiation, the polymeric material is irradiated in a solid state at a dose of about 0.01 to 100 MRad (0.1 to 1000 kGy), preferably from 1 to 20 MRad, using methods known in the art, such as exposure to gamma emissions from an isotope such as ⁶⁰Co. In various embodiments, γ-irradiation for a crosslinking is carried out at a dose of 1 to 20, preferably about 5 to 20 MRad. In a non-limiting embodiment, irradiation is to a dose of approximately 10 MRad.

Irradiation of the polymeric material is usually accomplished in an inert atmosphere or vacuum. For example, the polymeric material may be packaged in an oxygen impermeable package during the irradiation step. Inert gases, such as nitrogen, argon, and helium may also be used. When vacuum is used, the packaged material may be subjected to one or more cycles of flushing with an inert gas and applying the vacuum to eliminate oxygen from the package. Examples of package materials include metal foil pouches such as aluminum or Mylar® coating packaging foil, which are available commercially for heat sealed vacuum packaging. Irradiating the polymeric material in an inert atmosphere reduces the effect of oxidation and the accompanying chain scission reactions that can occur during irradiation. Oxidation caused by oxygen present in the atmosphere present in the irradiation is generally limited to the surface of the polymeric material. In general, low levels of surface oxidation can be tolerated as the oxidized surface can be removed during subsequent machining.

Irradiation such as γ-irradiation can be carried out on polymeric material at specialized installations possessing suitable irradiation equipment. When the irradiation is carried out at a location other than the one in which the further heating, doping, and machining operations are to be carried out, the irradiated material is conveniently left in the oxygen impermeable packaging during shipment to the site for further operations.

### Antioxidants

Antioxidant compositions useful herein contain one or more antioxidant compounds. Non-limiting examples of antioxidant compounds include tocopherols such as Vitamin E, carotenoids, triazines, Vitamin K, and others. Preferably, the antioxidant composition comprises at least about 10% of one or more antioxidant compounds. In various embodiments, the antioxidant composition is at least 50% by weight antioxidant up to an including 100%, or neat antioxidant.

As used here, the term Vitamin E is used as a generic descriptor for all tocol and tocotrienol derivatives that exhibit Vitamin E activity or the biological activity of α-tocopherol. Commercially, Vitamin E antioxidants are sold as Vitamin E, α-tocopherol, and related compounds. The term tocol is the trivial designation for 2-methyl-2-(4,8,12-trimethyltridecyl)chroman-6-ol (compound I, R¹═R²═R³═H).

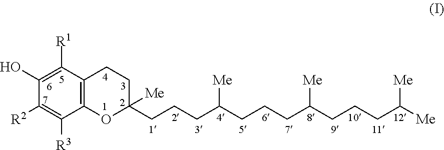

The term tocopherol is used as a generic descriptor for mono, di, and tri substituted tocols. For example, α-tocopherol is compound I where R¹═R²═R³=Me; β-tocopherol is compound I where R¹═R³=Me and R²═H. Similarly, γ-tocopherol and δ-tocopherol have other substitution patterns of methyl groups on the chroman-ol ring.

Tocotrienol is the trivial designation of 2-methyl-2-(4,8,12-trimethyltrideca-3,7,11-trienyl)chroman-6-ol.

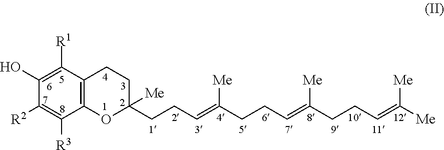

Examples of compound II include 5,7,8-trimethyltocotrienol, 5,8-dimethyltocotrienol, 7,8-dimethyltocotrienol, and 8-methyltocotrienol.

In compound I, there are asymmetric centers at positions 2, 4′, and 8′. According to the synthetic or natural origin of the various tocol derivatives, the asymmetric centers take on R, S, or racemic configurations. Accordingly, a variety of optical isomers and diasteromers are possible based on the above structure. To illustrate, the naturally occurring stereoisomer of α-tocopherol has the configuration 2R, 4′R, 8′R, leading to a semi-systematic name of (2R,4′R,8′R)-α-tocopherol. The same system can be applied to the other individual stereoisomers of the tocopherols. Further information on Vitamin E and its derivatives can be found in book form or on the web published by the International Union of Pure and Applied Chemistry (IUPAC). See for example, 1981 recommendations on “Nomenclature of Tocopherols and Related Compounds.”

Carotenoids are a class of hydrocarbons (carotenes) and their oxygenated derivatives (xanthophylls) consisting of eight isoprenoid units joined in such a manner that the arrangement of isoprenoid units is reversed at the center of the molecule. As a result, the two central methyl groups are in a 1,6-positional relationship and the remaining nonterminal methyl groups are in a 1,5-positional relationship. The carotenoids are formally derived from an acyclic C₄₀H₅₆ structure having a long central chain of conjugated double bonds. The carotenoid structures are derived by hydrogenation, dehydrogenation, cyclization, or oxidation, or any combination of these processes. Specific names are based on the name carotene, which corresponds to the structure and numbering shown in compound III.

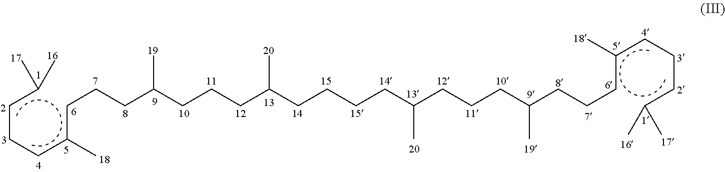

The broken lines at the two terminations represent two “double bond equivalents.” Individual carotene compounds may have C₉ acyclic end groups with two double bonds at positions 1,2 and 5,6 (IV) or cyclic groups (such as V, VI, VII, VIII, IX, and X).

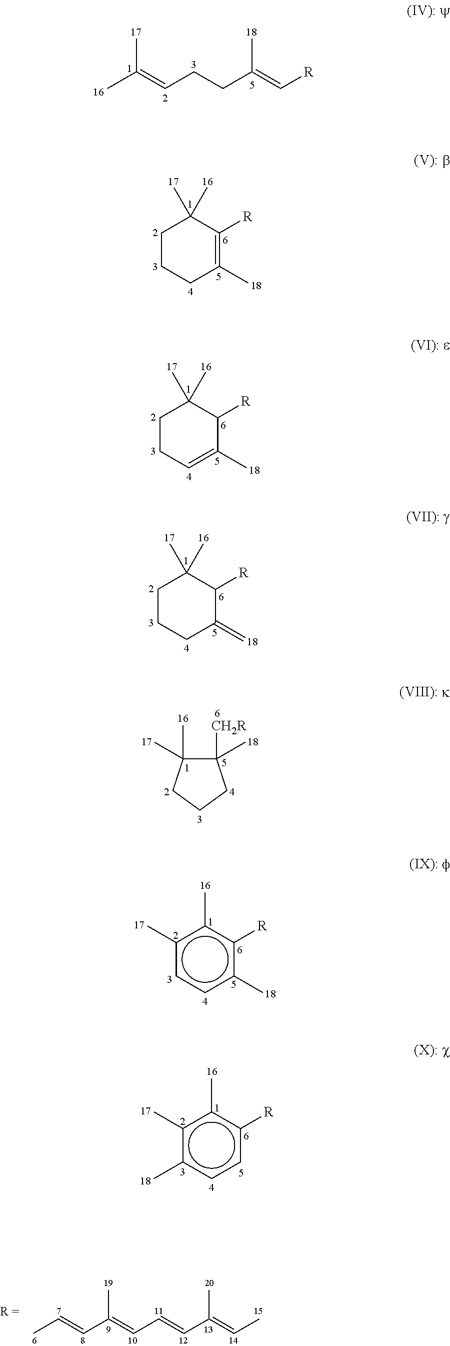

The name of a specific carotenoid hydrocarbon is constructed by adding two Greek letters as prefixes to the stem name carotene. If the end group is acyclic, the prefix is psi (ψ), corresponding to structure IV. If the end group is a cyclohexene, the prefix is beta (β) or epsilon (ε), corresponding to structure V or VI, respectively. If the end group is methylenecyclohexane, the designation is gamma (γ), corresponding to structure VII. If the end group is cyclopentane, the designation is kappa (κ), corresponding to structure VIII. If the end group is aryl, the designation is phi (φ) or chi (χ), corresponding to structures IX and X, respectively. To illustrate, “β-carotene” is a trivial name given to asymmetrical carotenoid having beta groups (structure V) on both ends.

Elimination of a CH₃, CH₂, or CH group from a carotenoid is indicated by the prefix “nor”, while fusion of the bond between two adjacent carbon atoms (other than carbon atoms 1 and 6 of a cyclic end group) with addition of one or more hydrogen atoms at each terminal group thus created is indicated by the prefix “seco”. Furthermore, carotenoid hydrocarbons differing in hydrogenation level are named by use of the prefixes “hydro” and “dehydro” together with locants specifying the carbon atoms at which hydrogen atoms have been added or removed.

Xanthophylls are oxygenated derivatives of carotenoid hydrocarbons. Oxygenated derivatives include without limitation carboxylic acids, esters, aldehydes, ketones, alcohols, esters of carotenoid alcohol, and epoxies. Other compounds can be formally derived from a carotenoid hydrocarbon by the addition of elements of water (H, OH), or of alcohols (H, OR, where R is C₁₋₆ alkyl) to a double bond.

Carotenoids having antioxidant properties are among compounds suitable for the antioxidant compositions of the invention. Non-limiting examples of the invention include Vitamin A, retinoids and beta-carotene.

Other antioxidants include Vitamin C (absorbic acid) and its derivatives; Vitamin K; gallate esters such propyl, octyl, and dodecyl; lactic acid and its esters; tartaric acid and its salts and esters; and ortho phosphates. Further non-limiting examples include polymeric antioxidants such as members of the classes of phenols; aromatic amines; and salts and condensation products of amines or amino phenols with aldehydes, ketones, and thio compounds. Non-limiting examples include para-phenylene diamines and diaryl amines.

Antioxidant compositions preferably have at least 10% by weight of the antioxidant compound or compounds described above. In preferred embodiments, the concentration is 20% by weight or more or 50% by weight or more. In various embodiments, the antioxidant compositions are provided dissolved in suitable solvents. Solvents include organic solvents and supercritical solvents such as supercritical carbon dioxide. In other embodiments, the antioxidant compositions contain emulsifiers, especially in an aqueous system. An example is Vitamin E (in various forms such as α-tocopherol), water, and suitable surfactants or emulsifiers. In a preferred embodiment, when the antioxidant compound is a liquid, the antioxidant composition consists of the neat compounds, or 100% by weight antioxidant compound.

### Doping

In various embodiments, the antioxidant composition is doped into the crosslinked polymeric material to provide an antioxidant at an effective level. Preferably, the methods provide a rapid method of doping to provide effective antioxidant levels at decreased times.

During the doping process, the crosslinked polymer material is exposed to antioxidant in a doping step. In various embodiments, the crosslinked polymer is contacted with a liquid composition including an antioxidant. The contacting provides an intermediate crosslinked polymer with the antioxidant on its surface. By “contacted” or “contacting,” it is meant that the crosslinked polymer is in close proximity with, or touching, the antioxidant. In various embodiments, the crosslinked polymer is soaked in a liquid composition including the antioxidant. In various embodiments, at least part of the crosslinked polymer is immersed in the liquid composition comprising the antioxidant. Alternatively or in addition, the antioxidant composition is applied to the surface of the polymer by other means such as dipping, spraying, wiping, brushing, painting, and the like. Total exposure time of the polymer material to the antioxidant is selected to achieve suitable penetration of the antioxidant. In various embodiments, total exposure time is at least several hours and preferably greater than or equal to one day (24 hours).

The temperature and pressure conditions of exposing the crosslinked polymer material to the antioxidant composition are preferably those at which the composition remains a liquid. Lower temperatures tend to retard or mitigate unwanted oxidation of the polymer material, especially in doping conditions that do not exclude oxygen. If the exposure conditions exclude oxygen, then the temperature can be elevated if desired to achieve faster doping times.

Doping with antioxidant is preferably carried out at a temperature at which the time required for doping is commercially reasonable. In a typical embodiment, the temperature is above room temperature and preferably above 50° C., above 60° C., above 70° C., or above 80° C. In a preferred embodiment, especially when the antioxidant is vitamin E, the temperature is 90° C. or higher. Doping is preferably carried out at a temperature below the onset melting temperature of the polymer being doped. At ambient conditions this means below about 135 or 136° C. when the polymer is ultrahigh molecular weight polyethylene. For UHMWPE, a range of 120-130° C. is suitable, being high enough for the rate of in-diffusion to be acceptable but not so high that the polymer properties are lost by heating above an onset melting temperature.

Pressure can be applied during the doping step during which the polymer is contacted with or exposed to an antioxidant composition. That is, the pressure can be higher than one atmosphere during the time the polymer is exposed to the liquid composition including the antioxidant. In various embodiments, pressure is applied to a fluid (liquid composition) in which the polymer is immersed. Pressure and temperature conditions can be selected in consideration of the atmosphere otherwise present to provide suitable doping results. In one embodiment, temperature and pressure are ambient (i.e. atmospheric pressure and room temperature). The temperature can be less than or higher than room temperature (but is preferably elevated above room temperature); the pressure can be elevated, or any combination.

In some embodiments, the doping or exposure to antioxidant is carried out at high pressures and/or under temperature conditions such as those described below for the subsequent homogenizing step. In preferred embodiments, at least one of the doping and the homogenizing steps is carried out at high pressure and elevated temperature. As to doping, the application of high pressure and/or elevated temperatures leads to more complete or faster incorporation of the antioxidant into the interior of the polymer.

After doping, the polymer may be removed from contact with the antioxidant. For example, if it was immersed, the polymer is removed from the liquid and the antioxidant wiped off or allowed to drip off. After removal from contact with the antioxidant, some residual antioxidant remains on the outside surface of the polymer, even if it was wiped off. Alternatively, the polymer, after removal, has antioxidant diffused into at least the surface portion of the bulk of the material, but not completely diffused into the interior. In either case, the polymer is said to have antioxidant on its surface. In this and other embodiments, it is understood that removing the polymer such as UHMWPE from exposure to the antioxidant composition encompasses both removing the polymer physically from the composition and removing the composition while leaving the bar in place, such as by decanting, siphoning, draining, or pouring, by way of non-limiting example. Combinations of the two methods may also be used. It is further understood that exposing the polymer material to the antioxidant composition can involve both plunging the crosslinked polymer material into the composition and pouring the composition onto the bar to cover it. As before, combinations of the two may also be used.

This intermediate is then further treated by the homogenizing steps.

### Homogenizing

The doping step is followed by a subsequent homogenizing step. This step is desirably carried out at an elevated temperature to speed up the process by which antioxidant diffuses into the polymer. Preferably, the temperature of homogenizing is below the onset melting temperature of the polymer, in order to maintain the strength and other properties at desirable levels. In an advance described herein, an elevated pressure is applied during the homogenizing step. The applied pressure is sufficiently elevated that it affects and raises the melting temperature of the polymer. Application of the high pressure shifts the onset melting temperature of the polymer as well as its peak melting temperature to higher values than those obtaining at ambient conditions of atmospheric pressure. The melting temperature shift is to an elevated onset melting temperature. The homogenizing is then carried out at a temperature higher than the normal or ambient onset melting temperature, but still below the elevated onset melting temperature of the polymer that exists at the pressures used in the homogenizing step.

In this way, the homogenizing temperature in the current technology can be higher than the normal onset melting temperature, which otherwise would set an upper limit for the temperature of homogenizing to avoid degrading the polymer by melting or partial melting. Instead, the upper limit is given by the elevated onset melting temperature, which is raised 5 or more degrees Celsius, in an exemplary embodiment, by the application of pressure during the homogenizing step. Raising the pressure extends the temperature upward at which the polymer can be heated without exceeding the temperature at which it would degrade due to melting.

In certain aspects, the crosslinked polymer is exposed to an elevated pressure in an inert atmosphere. An “inert atmosphere” refers to an environment with low levels of oxygen relative to air, for example an atmosphere with less than 1% oxygen, and preferably an essentially oxygen-free environment. An inert atmosphere has a decreased level of O₂ which would otherwise tend to oxidize the polymer material during the homogenizing process. In some embodiments, the inert atmosphere comprises an inert gas. In some embodiments, the inert atmosphere is selected from the group consisting of N₂, argon and CO₂.

In one aspect, it is desirable to provide methods of achieving a suitable level of antioxidant in the interior or inner portions of the polymer material, while avoiding excess antioxidant at the outer surface. In various embodiments, during the homgenizing process, the crosslinked polymeric material with the antioxidant on its surface is exposed to an elevated pressure in an inert atmosphere. As noted, in a preferred embodiment, the elevated pressure applied during homogenizing is high enough to raise the onset melting temperature and the crystalline melting point of the crosslinked polymer being so treated. In various embodiments, the elevated pressure is 10 MPa to 500 MPa or from 10 MPa to 100 MPa. In various embodiments, the elevated pressure is sufficient to raise the onset melting temperature by 5° C. or more above the ambient onset melting temperature of the crosslinked polymer, i.e. the normal onset melting temperature at atmospheric pressure.

Furthermore, without limiting the scope, function or utility of the present technology, it is believed that the method of exposing the crosslinked polymer to an elevated pressure in an inert atmosphere to raise the onset melting temperature of the polymer lowers the diffusion time of antioxidant into the interior of the crosslinked polymer, such as UHMWPE. Conventionally, the diffusion process for UHMWPE has been limited to a temperature of about 130° C. or not over about 135° C. because going over this temperature ran the risk of melting the UHMWPE and reducing its mechanical properties. As noted, this upper limit of temperature was imposed so as not to heat the polymer above the onset melting temperature. When pressure is applied to the UHMWPE as in the current technology, the temperature at which the polymer melts also increase. The UHMWPE with an antioxidant such as Vitamin E on the surface is exposed to an inert atmosphere under an elevated pressure, which in turns raises the melting temperature. Because the melting temperature of the polymer is higher at elevated pressure, the homogenizing temperature can be increased at the elevated pressure without affecting the mechanical properties of the UHMWPE. The higher homogenizing temperature in turn reduces the times required to diffuse Vitamin E through the full thickness of the UHMWPE. In various embodiments, UHMWPE is exposed and homogenized at a temperature greater than about 130° C.

During the homogenizing step, the antioxidant continues to diffuse into the interior of the polymer material. In various embodiments, the homogenizing process occurs in a time sufficient to achieve diffusion of the antioxidant from the surface into the interior of the polymer. In various embodiments, the total time of homogenizing is at least several hours and can be more than one day. For example, while there is no particular upper limit, homogenizing is preferably carried out for at least an hour after doping, and typically for a period from about 1 to about 600 hours, from about 5 to about 400 hours, or from about 10 to about 100 hours. Depending on the size of the part, the post doping heating may be carried out for a period of from about 10 to about 14 days, or from about 11 to about 19 days, by way of non-limiting example.

### Optional Sequential Doping and Homogenizing

In various embodiments, the doping and homogenizing steps can be repeated as desired to achieve suitable diffusion of the antioxidant through the polymer material. During a sequential doping process, the crosslinked polymer material is exposed to antioxidant at least two times, with a homogenizing step in between the times of exposure. Total exposure time of the crosslinked polymer material to the antioxidant is selected to achieve suitable penetration of the antioxidant. In various embodiments, total exposure time is at least several hours and preferably greater than or equal to one day (24 hours).

In between times of exposure of the crosslinked polymer material to antioxidant, the crosslinked polymer material is homogenized by subjecting the crosslinked polymer material to an elevated pressure in an inert atmosphere wherein the elevated pressure is high enough to raise the melting temperature as described herein. The crosslinked polymer material is also heated at the elevated pressure to a temperature above the normal (or ambient) onset melting temperature but below the elevated onset melting temperature for a sufficient time to allow diffusion of the antioxidant. During the homogenizing step, the antioxidant continues to diffuse into the interior of the crosslinked polymer material. With sequential doping and homogenizing, the times of homogenizing are broken up into two or more steps, with the total time being preferably at least several hours and more preferably more than one day.

In various embodiments, breaking up the time of exposure to antioxidant and the time of homogenizing into two or more periods provides greater diffusion of the antioxidant into the interior of the polymer material than the same amount of time of exposure in one dose. At the same time, the method tends to avoid an accumulation of antioxidant on the surface of the polymer material, which could lead to undesirable exudation or “sweating” of the polymer material, as excess antioxidant rises to the surface and escapes from the polymer. Furthermore, without limiting the scope, function or utility of the present technology, it is believed that the sequential doping method provides additional “driving force” for the diffusion of antioxidant into the interior of the polymer material. The driving force is proportional to the concentration difference or gradient of the antioxidant such as α-tocopherol on the surface and inside the polymer of the polymeric material. As the antioxidant diffuses into the polymer, the driving force is reduced. In various embodiments, the methods of the invention counteract the reduced driving force by recharging it periodically with sequential doping of the antioxidant.

In various embodiments, the sequence of steps constituting a doping/removing/heating cycle is carried out 2, 3, 4, or more times as desired to provide the desired level of doping of antioxidant. Preferably, the total time of exposure of the polymeric polymer material to the antioxidant during the plurality of doping cycles is at least several hours, preferably greater than one day and preferably greater than two days, up to 3 weeks, 2 weeks, or one week when held for example at about 130° C. The total time of homogenizing when out of contact with the antioxidant composition is preferably at least several hours over the plurality of cycles. Preferably, the homogenizing time is greater than one day and preferably greater than two days, up to one week, two weeks, or three weeks of total homogenizing time during the cycles. During the homogenizing steps when out of contact, the antioxidant further diffuses into the interior of the polymer material.

In various embodiments, advantages of processes including the sequential doping steps described above are achieved even when the homogenizing is carried out conventionally at normal or ambient pressures and below the ambient melting point of the polymer.

### Machining to the Final Shape of the Implant Component

After the annealing process, in various embodiments, the polymer is cooled. A machining or other manufacturing step or steps is carried out to produce a polymer material in the shape of the ultimate bearing component. In one embodiment, the doped polymer is in the form of a bar or other bulk preform that is subsequently cut into billets and further processed to an implant such as an acetabular cup. If the polymer is in the form of a near net shape preform, the further processing steps are used to remove a fairly small amount of material, illustratively from about 1 to about 15 mm, from about 2 to about 10 mm, or from about 3 to about 4 mm from the polymer material that was crosslinked, and then doped with antioxidant and annealed. Advantageously, the dimensions of the polymer can be selected so that, depending on demand, a number of different implant components or sizes of implant components can be machined from the polymer material. Thus for example, it is possible to make and stockpile a supply of polymer materials, and produce implant components as needed in the sizes required. The machining step removes an outer surface or layer of the polymer material. This may provide the further advantage of removing an eluting outer layer of the polymer material that might have been produced during the doping and homogenizing steps.

Non-limiting examples of implant components include tibia bearings, acetabular linings, glenoid components of an artificial shoulder, and spinal components such as those used for disk replacement or in a motion preservation system.

### Products of the Methods

In various embodiments, the methods provide polymer materials especially in the form of a medical implant bearing components having significant levels of antioxidants throughout the interior of the polymer material. In a preferred embodiment, the implants have a level of antioxidant that is below the saturation level at which sweating or eluting of antioxidant would be observed.

In general, the free radical concentration in the polymer changes as the various process steps are carried out. The consolidated UHMWPE starting material and the nascent UHMWPE powder contain essentially no free radicals. The unirradiated polymer materials likewise have essentially no detectable free radicals. On crosslinking, the free radical concentration grows to a measurable level, which is slightly reduced when the irradiated polymer material is doped with antioxidant. The level of detectable free radicals is further significantly reduced during the post doping heat treatment or homogenizing step. The final machining step has little effect on free radicals, while the final irradiation sterilization increases free radicals slightly. Non-irradiative sterilization has no effect on free radicals. But throughout, the free radicals are not reduced to non-detectable levels at any time after the irradiation. This is in contrast to crosslinked materials that have been heated or even melted to recombine free radicals and reduce their concentration. But despite the relatively higher concentration of free radicals, antioxidant-doped crosslinked polymers of the invention maintain a high resistance to oxidation, which, without limiting the scope, function or utility of the present technology, is believed to be attributable to a sequestration of the free radicals in close association with the antioxidant compounds.

It has been found that UHMWPE, preforms, and bearing components made according to the invention have a high level of oxidative resistance, even though free radicals can be detected in the bulk material. To measure and quantify oxidative resistance of polymeric materials, it is common in the art to determine an oxidation index by infrared methods such as those based on ASTM F 2102-01. In the ASTM method, an oxidation peak area is integrated below the carbonyl peak between 1650 cm⁻¹ and 1850 cm⁻¹. The oxidation peak area is then normalized using the integrated area below the methylene stretch between 1330 cm⁻¹ and 1396 cm⁻¹. Oxidation index is calculated by dividing the oxidation peak area by the normalization peak area. The normalization peak area accounts for variations due to the thickness of the sample and the like. Oxidative stability can then be expressed by a change in oxidation index upon accelerated aging. Alternatively, stability can be expressed as the value of oxidation attained after a certain exposure, since the oxidation index at the beginning of exposure is close to zero. In various embodiments, the oxidation index of crosslinked polymers of the invention changes by less than 0.5 after exposure at 70° C. to five atmospheres oxygen for four days. In preferred embodiments, the oxidation index shows a change of 0.2 or less, or shows essentially no change upon exposure to five atmospheres oxygen for four days. In a non-limiting example, the oxidation index reaches a value no higher than 1.0, preferably no higher than about 0.5, after two weeks of exposure to 5 atm oxygen at 70° C. In a preferred embodiment, the oxidation index attains a value no higher than 0.2 after two or after four weeks exposure at 70° C. to 5 atm oxygen, and preferably no higher than 0.1. In a particularly preferred embodiment, the specimen shows essentially no oxidation in the infrared spectrum (i.e. no development of carbonyl bands) during a two week or four week exposure. In interpreting the oxidative stability of UHMWPE prepared by these methods, it is to be kept in mind that the background noise or starting value in the oxidation index determination is sometimes on the order of 0.1 or 0.2, which may reflect background noise or a slight amount of oxidation in the starting material.

In various embodiments, implant bearing components are manufactured from polymeric starting materials using the methods described herein. Non-limiting examples of bearing components include those in hip joints, knee joints, ankle joints, elbow joints, shoulder joints, spine, temporo-mandibular joints, and finger joints. In hip joints, for example, the methods can be used to make the acetabular cup or the insert or liner of the cup. In the knee joints, the compositions can be made used to make the tibial plateau, the patellar button, and trunnion or other bearing components depending on the design of the joints. In the ankle joint, the compositions can be used to make the talar surface and other bearing components. In the elbow joint, the compositions can be used to make the radio-numeral or ulno-humeral joint and other bearing components. In the shoulder joint, the compositions can be used to make the glenero-humeral articulation and other bearing components. In the spine, intervertebral disc replacements and facet joint replacements may be made from the compositions.

The methods described herein provide additional benefits to the manufacturing process. When doping is carried out on a finished component, growth and shrinkage of the UHMWPE observed upon addition of antioxidant can cause the geometry to change significantly. On the other hand, machining the final component from a near net shape polymer material as described herein produces a product that is dimensionally accurate and dimensionally stable. The machining step thus eliminates a variable and makes the process more predictable.

### Non-Limiting Discussion of Terminology

The headings (such as “Introduction” and “Summary”) and sub-headings used herein are intended only for general organization of topics within the present disclosure, and are not intended to limit the disclosure of the technology or any aspect thereof. In particular, subject matter disclosed in the “Introduction” may include novel technology and may not constitute a recitation of prior art. Subject matter disclosed in the “Summary” is not an exhaustive or complete disclosure of the entire scope of the technology or any embodiments thereof. Classification or discussion of a material within a section of this specification as having a particular utility is made for convenience, and no inference should be drawn that the material must necessarily or solely function in accordance with its classification herein when it is used in any given composition or method.

The citation of references herein does not constitute an admission that those references are prior art or have any relevance to the patentability of the technology disclosed herein. Any discussion of the content of references cited in the Introduction is intended merely to provide a general summary of assertions made by the authors of the references, and does not constitute an admission as to the accuracy of the content of such references. All references cited in the “Description” section of this specification are hereby incorporated by reference in their entirety.

The description and specific examples, while indicating embodiments of the technology, are intended for purposes of illustration only and are not intended to limit the scope of the technology. Moreover, recitation of multiple embodiments having stated features is not intended to exclude other embodiments having additional features, or other embodiments incorporating different combinations of the stated features. Specific examples are provided for illustrative purposes of how to make and use the compositions and methods of this technology and, unless explicitly stated otherwise, are not intended to be a representation that given embodiments of this technology have, or have not, been made or tested. Equivalent changes, modifications and variations of embodiments, materials, compositions and methods can be made within the scope of the present technology, with substantially similar results.

As used herein, the words “desire” or “desirable” refer to embodiments of the technology that afford certain benefits, under certain circumstances. However, other embodiments may also be desirable, under the same or other circumstances. Furthermore, the recitation of one or more desired embodiments does not imply that other embodiments are not useful, and is not intended to exclude other embodiments from the scope of the technology.

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of non-restrictive terms such as including, containing, or having, is used herein to describe and claim embodiments of the present technology, embodiments may alternatively be described using more limiting terms such as “consisting of or “consisting essentially of.” Thus, for any given embodiment reciting materials, components or process steps, the present technology also specifically includes embodiments consisting of, or consisting essentially of, such materials, components or processes excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, all compositional percentages are by weight of the total composition, unless otherwise specified. Disclosures of ranges are, unless specified otherwise, inclusive of endpoints and include disclosure of all distinct values and further divided ranges within the entire range. Thus, for example, a range of “from A to B” or “from about A to about B” is inclusive of A and of B. Disclosure of values and ranges of values for specific parameters (such as temperatures, molecular weights, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, and 3-9.

When an element or layer is referred to as being “on”, “engaged to”, “connected to” or “coupled to” another element or layer, it may be directly on, engaged, connected or coupled to the other element or layer, or intervening elements or layers may be present. In contrast, when an element is referred to as being “directly on”, “directly engaged to”, “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

“Melting point” and “melting temperature” are used interchangeably. “Ambient” pressure refers to about one atmosphere. The “normal” or “ambient” melting temperature or melting point of a crosslinked polymer is its melting temperature measured at ambient pressure.

What is claimed is:

1. A method for preparing an antioxidant doped polymer, the method comprising:
(a) contacting a crosslinked polymer with a liquid composition comprising an antioxidant, to provide an intermediate polymer with the antioxidant on its surface;
(b) homogenizing the intermediate polymer by:
(1) exposing the intermediate polymer to an elevated pressure in an inert atmosphere, wherein the elevated pressure is high enough to raise the onset melting temperature of the intermediate polymer to an elevated onset melting temperature above an ambient onset melting temperature determined at atmospheric pressure; and
(2) heating the intermediate polymer at the elevated pressure to a temperature above the ambient onset melting temperature but below the elevated onset melting temperature for a time sufficient to achieve diffusion of the antioxidant from the surface into the interior of the intermediate polymer and produce a doped polymer; and
(c) cooling the doped polymer after homogenizing.

2. A method according to claim 1, wherein the crosslinked polymer is in the form of a cylindrical rod.

3. A method according to claim 1, wherein the antioxidant is selected from the group consisting of α-tocopherol, retinoids, Vitamin E, and mixtures thereof.

4. A method according to claim 3, wherein the antioxidant composition comprises Vitamin E.

5. A method according to claim 1, wherein the elevated pressure is 10 MPa to 500 MPa.

6. A method according to claim 5, wherein the elevated pressure is 10 to 100 MPa.

7. A method according to claim 1, wherein the elevated pressure is sufficient to raise the onset melting temperature of the intermediate polymer to an elevated onset melting temperature that is least 5° C. greater than the ambient onset melting temperature.

8. A method according to claim 1, wherein the contacting comprises immersing at least part of the crosslinked polymer in the liquid composition.

9. A method according to claim 8, wherein the liquid composition comprises neat antioxidant.

10. A method according to claim 1, wherein the contacting is carried out at an elevated pressure and below the onset melting temperature of the intermediate polymer.

11. A method according to claim 7, comprising heating the intermediate polymer at a temperature at least 5° C. greater than the ambient onset melting temperature.

12. A method according to claim 1, comprising repeating steps (a) and (b) through (d) at least once to achieve a desired level of antioxidant doping.

13. A method according to claim 1, further comprising machining an implant bearing component from the cooled doped polymer.

14. A method according to claim 1, wherein the inert atmosphere comprises an inert gas.

15. A method according to claim 1, wherein the inert atmosphere is selected from the group consisting of N₂, argon, and CO₂.

16. A method of preparing antioxidant doped UHMWPE, comprising:
(a) doping a crosslinked UHMWPE by contacting with a liquid composition comprising Vitamin E to produce a UHMWPE intermediate with Vitamin E on its surface;
(b) homogenizing the intermediate UHMWPE by:
(1) subjecting the intermediate UHMWPE with Vitamin E on its surface to a pressure above 10 MPa, at which the pressure the intermediate UHMWPE has an elevated onset melting point higher than its onset melting point at atmospheric pressure; and
(2) heating the intermediate UHMWPE at the elevated pressure to a temperature greater than the onset melting point at atmospheric pressure but less than the elevated onset melting point for a time sufficient to achieve diffusion of Vitamin E from the surface to the interior of the intermediate UHMWPE; and
(c) cooling the UHMWPE after homogenizing.

17. A method according to claim 16, wherein the liquid composition is neat Vitamin E.

18. A method according to claim 16, wherein the homogenizing is carried out with the crosslinked UHMWPE at least partially immersed in the liquid composition.

19. A method according to claim 16, comprising subjecting the intermediate UHMWPE to a pressure of 10 to 500 MPa.

20. A method according to claim 16, wherein the pressure is sufficient to achieve an elevated onset melting point at least 5° C. greater than the ambient onset melting point.

21. A method according to claim 16, wherein the crosslinked UHMWPE is in the form of a rod having a diameter of 2 to 4 inches.

22. A method according to claim 16, wherein the intermediate UHMWPE is a near shape bearing component of an artificial joint.

23. A method according to claim 16, comprising repeating steps (a) and (b) to achieve a desired level of incorporation of Vitamin E in the intermediate UHMWPE.

24. A method according to claim 16, further comprising machining a bearing component from the cooled doped UHMWPE.

25. A method of making an artificial joint component, comprising:
(a) doping a crosslinked UHMWPE by contacting it with a liquid composition comprising Vitamin E to produce a UHMWPE intermediate with Vitamin E on its surface;
(b) homogenizing the intermediate UHMWPE by:
(1) subjecting the intermediate UHMWPE with Vitamin E on its surface to a pressure above 10 MPa, at which pressure the intermediate UHMWPE has an elevated onset melting point higher than its onset melting point at atmospheric pressure; and
(2) heating the intermediate UHMWPE at the elevated pressure to a temperature greater than the onset melting point at atmospheric pressure but less than the elevated onset melting point for a time sufficient to achieve diffusion of Vitamin E from the surface to the interior of the intermediate UHMWPE;
(c) cooling the doped UHMWPE after homogenizing; and
(d) machining the joint component from the doped UHMWPE treated by steps (a) through (c).

26. A method according to claim 25, comprising subjecting the intermediate UHMWPE to a pressure of 10 to 500 MPa.

27. A method according to claim 25, wherein the pressure is sufficient to achieve an elevated onset melting point at least 5° C. greater than the ambient onset melting point.

28. A method according to claim 25, comprising repeating steps (a) and (b) to achieve a desired level of incorporation of Vitamin E in the intermediate UHMWPE.
